(19) United States
(12) Patent Application Publication
(10) Pub. No.: US 2026/0158271 A1
(43) Pub. Date: Jun. 11, 2026

(54) MYOELECTRIC SENSING FOR PRECISION ELECTRICAL MUSCLE STIMULATION

(71) Applicant: Hillmed Inc., Katy, TX (US)

(72) Inventors: Yingchun Zhang, Coral Gables, FL (US); Nicholas Dias, Houston, TX (US)

(21) Appl. No.: 19/410,040

(22) Filed: Dec. 5, 2025

Related U.S. Application Data
(60) Provisional application No. 63/728,500, filed on Dec. 5, 2024.

(51) Int. Cl. A61N 1/36; A61M 5/42; A61N 1/04

(52) U.S. Cl. CPC A61N 1/36031; A61M 5/427; A61N 1/0452; A61N 1/0476; A61N 1/36003; A61M 2205/054; A61M 2230/60

(57) ABSTRACT

An electrode array system for electromyography-guided therapeutic intervention comprises a flexible substrate with a plurality of electrodes arranged in a structured array configuration and a plurality of voids extending through the substrate. The electrodes detect electromyographic signals from underlying musculature, which are transmitted to a signal acquisition and therapy module via conductive traces and a wired connection interface. The signal acquisition and therapy module processes the electromyographic signals to identify motor unit territories exhibiting pathological activity. The voids are strategically positioned between adjacent rows of electrodes to provide unobstructed access for syringe-mediated delivery of therapeutic agents, such as neurolytic agents, directly to the identified motor unit territories. The system generates spatial maps, including root mean square heatmaps and innervation zone point clouds, to guide precise injection coordinates. The integration of high-density surface electromyography with injection guidance enables functionally targeted therapeutic delivery, maximizing treatment efficacy while minimizing dosage requirements and off-target effects.

### CROSS-REFERENCE TO RELATED APPLICATIONS

This application claims the benefit of priority under 35 U.S.C. § 119(e) to U.S. Provisional Patent Application No. 63/728,500, filed Dec. 5, 2024, entitled “ELECTROMYOGRAPHY-GUIDED INJECTION OF NEUROLYTIC AGENTS USING A FLEXIBLE ELECTRODE ARRAY,” the entire disclosure of which is hereby incorporated by reference herein.

### TECHNICAL FIELD

The present disclosure relates generally to systems and methods for guided therapeutic interventions in neuromuscular disorders, and more specifically to electrode array-based systems configured for high-density electromyographic signal acquisition, spatial mapping of motor unit territories, and precision-guided delivery of therapeutic agents and electrical stimulation to targeted neuromuscular regions.

### BACKGROUND

Effective treatment of neuromuscular disorders, such as spasticity, dystonia, chronic pain syndromes, and muscle weakness, often requires precise targeting of specific motor unit territories within affected muscles. Conventional techniques for delivering neurolytic agents, including blind injections and ultrasound-guided injections, lack the functional resolution necessary to reliably identify and target individual motor unit territories and neuromuscular junction regions. Blind injection techniques rely on anatomical landmarks without functional feedback, while ultrasound guidance visualizes anatomical structures but cannot detect or map the electrical activity patterns that define motor unit territories. Similarly, conventional electrical stimulation approaches suffer from limitations in spatial precision and functional targeting, delivering diffuse stimulation to bulk muscle tissue rather than targeted therapy to specific motor unit territories. As a result, these approaches lead to inconsistent therapeutic outcomes, suboptimal efficacy, excessive dosage requirements, and increased risk of off-target effects on adjacent healthy muscle tissue.

The complexity of muscle architecture, combined with variability in individual anatomy and motor unit organization, further complicates accurate therapeutic delivery. Muscles may exhibit heterogeneous patterns of hyperactivity or weakness across different motor unit territories, requiring spatially resolved assessment and targeted intervention. Current clinical tools lack the capability to simultaneously map functional motor unit activity and guide therapeutic delivery to identified regions, whether through injection of neurolytic agents or application of electrical stimulation.

### SUMMARY

Embodiments of the present disclosure provide systems and methods for electromyography-guided therapeutic intervention that integrate high-density surface electromyography with precision-guided delivery of therapeutic agents and electrical stimulation. The disclosed systems enable functionally targeted interventions based on spatially resolved mapping of motor unit territories, thereby improving treatment efficacy while minimizing off-target effects and reducing dosage requirements.

In one aspect, an electrode array for electromyography-guided therapeutic intervention comprises a flexible substrate fabricated from a biocompatible polymeric material, such as thermoplastic urethane (TPU), thermoplastic elastomer (TPE), polyester, or polyimide. The flexible substrate may have a generally rectangular shape with rounded corners and a thickness ranging from approximately 0.1 mm to 2 mm. The flexible substrate further comprises an adhesive backing configured to secure the electrode array to a skin surface, where the adhesive backing may comprise a biocompatible adhesive selected from at least one of a hydrogel adhesive, an acrylic adhesive, or a silicone-based adhesive.

A plurality of electrodes are disposed on the flexible substrate and arranged in a structured array configuration, such as an 8 by 8 array, an 8 by 10 array, or an 8 by 12 array of electrodes. In various embodiments, the plurality of electrodes comprises between 64 and 128 electrodes. The plurality of electrodes are spatially distributed on the flexible substrate in rows and columns at intervals ranging from approximately 5 mm to 15 mm, where the intervals correspond to a sampling frequency less than or equal to a Nyquist frequency of target muscle signals. This spatial distribution enables high-resolution detection of motor unit action potentials and prevents spatial aliasing of electromyographic signals. Each electrode comprises a conductive contact pad constructed of a material including at least one of gold, silver, silver/silver chloride (Ag/AgCl), stainless steel, or platinum/iridium (Pt/Ir), providing low-impedance electrical contact with the skin surface.

A plurality of conductive traces are deposited on or embedded within the flexible substrate, where the conductive traces are fabricated from an electrically conductive material comprising at least one of copper, gold, platinum, or silver. Each conductive trace operably connects a corresponding electrode to a connection interface. A wired connection interface is positioned adjacent to a first edge of the flexible substrate, where the wired connection interface is configured to operably connect the conductive traces to a signal acquisition and therapy module. The wired connection interface may comprise a connector selected from at least one of a snap connector, a zero-insertion force (ZIF) connector, or a flexible printed circuit (FPC) connector. The conductive traces extend from the plurality of electrodes to the wired connection interface along the flexible substrate, enabling simultaneous signal acquisition from all electrodes in the array.

A distinguishing feature of the electrode array is the inclusion of a plurality of voids formed within the flexible substrate, where the voids extend completely through a thickness of the flexible substrate to provide unobstructed access for syringe-mediated delivery of therapeutic agents to underlying tissue. The plurality of voids are strategically arranged in one or more rows positioned between adjacent rows of electrodes, enabling precise needle placement guided by the spatially resolved electromyographic mapping. In various embodiments, the plurality of voids comprises at least 20 voids, and each void has a cross-sectional dimension ranging from approximately 1 mm to 5 mm to accommodate passage of a hypodermic needle while minimizing loss of structural integrity of the flexible substrate. This configuration allows therapeutic agents, such as botulinum toxin or other neurolytic agents, to be delivered directly to identified motor unit territories, neuromuscular junction regions, or motor endplate zones exhibiting pathological hyperactivity.

In another aspect, a system for electromyography-guided therapeutic intervention comprises the electrode array operably connected to a signal acquisition and therapy module via the wired connection interface. The signal acquisition and therapy module comprises an analog front end configured to receive electromyographic signals from the plurality of electrodes and to amplify the electromyographic signals with high gain and low noise characteristics. The amplified signals are transmitted to an analog-to-digital converter (ADC) configured to convert the amplified electromyographic signals into digital signals at a sampling rate sufficient to capture motor unit action potential waveforms without aliasing. A microcontroller unit (MCU) or field-programmable gate array (FPGA) is configured to process the digital signals and to generate therapy control commands. The signal acquisition and therapy module further comprises a communication interface configured to transmit the digital signals to a processor and to receive therapy parameters from the processor. The communication interface may comprise a wireless transceiver selected from at least one of a Bluetooth transceiver, a Wi-Fi radio, or a Zigbee module, enabling untethered operation of the electrode array. The signal acquisition and therapy module further comprises a power supply comprising a rechargeable battery to provide portable operation.

In embodiments configured for electrical stimulation therapy, the signal acquisition and therapy module further comprises a digital-to-analog converter (DAC) configured to convert digital therapy waveforms into analog therapy signals, and a current/voltage driver circuit configured to deliver the analog therapy signals through selected electrodes of the plurality of electrodes. This bidirectional capability enables the system to both sense electromyographic activity and deliver therapeutic electrical stimulation through the same electrode array, ensuring precise spatial alignment between the regions of detected pathological activity and the locations of therapeutic intervention. The electrical stimulation may comprise high-frequency blocking stimulation at frequencies ranging from 1 kHz to 10 kHz for inducing muscle relaxation, or modulated high-frequency stimulation with a low-frequency envelope at frequencies ranging from 10 Hz to 50 Hz for promoting muscle strengthening.

The system further comprises a processor configured to receive the digital signals from the signal acquisition and therapy module, process the digital signals to identify motor unit territories within a target muscle, and generate the therapy parameters based on the identified motor unit territories. The processor may employ signal processing algorithms including band-pass filtering, notch filtering, blind source separation, spatial filtering, and motor unit decomposition to isolate individual motor unit action potentials from the multi-channel electromyographic data. The processor is further configured to generate at least one of a root mean square (RMS) heatmap or an innervation zone point cloud based on the digital signals. The RMS heatmap provides a color-coded visualization of signal amplitude across the electrode array, highlighting regions of elevated neuromuscular activity that may correspond to hypertonic or spastic motor unit territories. The innervation zone point cloud identifies spatial locations where motor unit action potentials exhibit polarity reversals, indicating proximity to neuromuscular junction regions or motor endplate zones. These spatial maps guide the clinician in selecting optimal injection sites that correspond functionally to identified motor unit territories, rather than relying solely on anatomical landmarks or ultrasound-guided approximations.

In various embodiments, the system operates in a closed-loop configuration, where real-time electromyographic feedback continuously informs therapy delivery parameters. For example, the processor may monitor changes in motor unit recruitment patterns, firing rates, or spatial distribution of activity during therapy delivery, and may adjust stimulation parameters or recommend additional injection sites to optimize therapeutic outcomes. The integration of high-density surface electromyography with guided therapeutic delivery within a single, flexible electrode array platform represents a significant advancement over conventional approaches, enabling personalized, functionally targeted interventions for neuromuscular disorders including spasticity, dystonia, chronic pain syndromes, pelvic floor dysfunction, and muscle weakness.

A variety of additional inventive aspects will be set forth in the description that follows. The inventive aspects can relate to individual features and to combinations of features. It is to be understood that both the foregoing general description and the following detailed description are exemplary and explanatory only and are not restrictive of the broad inventive concepts upon which the embodiments disclosed herein are based.

### BRIEF DESCRIPTION OF THE DRAWINGS

The accompanying drawings, which are incorporated in and constitute a part of this description, illustrate various aspects of the present disclosure. A brief description of the drawings is as follows:

FIG. 1 is an illustration of an exemplary neuromuscular modulation system configured for both monitoring and therapeutic intervention of neuromuscular activity.

FIG. 2 is a plan view of a flexible electrode array aligned with muscle fibers in a parallel orientation to the EMG electrode array.

FIG. 3 is a side profile view of the flexible electrode array shown in FIG. 2.

FIG. 4 is an alternate side profile view of the flexible electrode array of FIG. 2.

FIG. 5 is a perspective view of the flexible electrode array illustrated in FIG. 2.

FIG. 6 provides a close-up view of the flexible electrode array of FIG. 5, showing the flexible substrate with conductive electrodes and voids configured for injection.

FIG. 7 is an illustration of an exemplary configuration in which the electrode array is positioned externally on the arm of a patient to detect muscle activity and deliver electrical stimulation.

FIG. 8 is an illustration of an exemplary testing protocol for evaluating neuromuscular activity and assessing treatment efficacy using the neuromuscular modulation system.

FIG. 9 is an illustration of an exemplary process of EMG decomposition as performed by the neuromuscular modulation system.

FIG. 10 is an illustration depicting an exemplary location root mean square (RMS) heatmap of the motor endplate territories generated from high-density EMG signal analysis highlighting regions of high neuromuscular activity intensity for targeted therapeutic delivery.

FIG. 11 illustrates innervation zone (IZ) point clouds, further refining the mapping shown in FIG. 10 by demonstrating a depth and spatial distribution of identified IZ regions to guide precise injection coordinates.

FIG. 12 is a flowchart depicting a method of using a system for electromyography (EMG)-guided injection via a flexible electrode array.

FIG. 13 illustrates example components of the signal acquisition and therapy module of FIG. 1.

FIG. 14 is an illustration of an embodiment of the processor or computing device of the system configured to provide processing and control functionality.

### DETAILED DESCRIPTION

Effective treatment of neuromuscular disorders, such as spasticity, dystonia, and chronic pain syndromes, often requires the precise targeting of specific motor unit territories within affected muscles for the delivery of therapeutic agents. Neurolytic agents, such as botulinum toxin, can be used to disrupt nerve function in hyperactive or dysfunctional neural pathways, providing relief from involuntary muscle contractions, pain, and functional impairment. However, conventional injection techniques, such as blind palpation-guided or ultrasound-guided injections, lack the ability to reliably identify and target individual motor unit territories at the neuromuscular junction level. These methods rely on anatomical landmarks or gross muscle visualization, which provide insufficient spatial resolution to distinguish between discrete motor endplate regions within a muscle. As a result, therapeutic agents may be delivered to suboptimal locations, leading to inconsistent clinical outcomes, reduced treatment efficacy, and an increased risk of complications, including unintended weakening of adjacent healthy muscle tissue.

The challenges associated with precise neuromuscular targeting are particularly pronounced in anatomically complex or deeply situated musculature, where surface landmarks provide limited guidance and ultrasound imaging may be constrained by tissue depth, acoustic shadowing, or operator skill. In addition, individual variability in muscle architecture, motor unit distribution, and innervation patterns further complicates the accurate delivery of therapeutic agents to the intended neuromuscular regions. Clinicians and specialists in fields such as neurology, physical medicine and rehabilitation, and pain management regularly face these challenges when treating conditions that require highly localized interventions. Despite advances in imaging and anatomical mapping, there remains a significant unmet need for techniques that can identify motor unit territories with high spatial and functional resolution, enabling clinicians to deliver neurolytic agents directly to the sites of pathological neuromuscular activity.

Embodiments of the present disclosure address these challenges by introducing a system and method that utilizes flexible electrode arrays configured for high-density electromyographic (EMG) monitoring and in-situ therapeutic delivery. The system employs a flexible substrate with an array of conductive traces and contact pads, spatially distributed to enable high-fidelity detection of motor unit action potentials across the surface of a target muscle. The electrode array further incorporates voids formed within the substrate, which are strategically positioned to facilitate syringe-mediated delivery of neurolytic agents directly through the array while preventing contamination of the injection site by substrate material. This integrated design enables the system to map motor unit territories based on spatial and temporal characteristics of detected EMG signals and subsequently guide the precise placement and delivery of therapeutic agents to those identified territories without the need to reposition or remove the electrode array.

The electrode array is operably connected to a signal acquisition and therapy module comprising an analog front end for signal amplification, a digitizer for analog-to-digital conversion, and a microcontroller unit (MCU) or field-programmable gate array (FPGA) for real-time signal processing. The system processes electromyographic signals using advanced techniques, including blind source separation, spatial filtering, and motor unit decomposition, to identify regions of interest within the musculature corresponding to discrete motor unit territories. These identified regions are visualized as root mean square (RMS) heatmaps or innervation zone point clouds, providing clinicians with spatially resolved, depth-encoded maps that guide injection coordinates with anatomical precision. By delivering neurolytic agents directly to motor endplate territories identified through functional EMG analysis, the system maximizes therapeutic precision and efficacy while minimizing off-target effects and reducing the dosage required to achieve clinical outcomes.

In addition to injection guidance, embodiments of the system can be configured to deliver electrical stimulation for neuromuscular modulation, enabling both diagnostic assessment and therapeutic intervention through a single integrated platform. The system supports predefined movement protocols in which subjects activate specific muscles, allowing the system to capture and analyze electromyographic signatures corresponding to the activation of target motor units. The integration of high-density surface EMG with advanced signal processing techniques, such as spatial filter bank construction and bioelectric source imaging, enables the system to determine not only the spatial location but also the depth of motor unit territories, providing comprehensive anatomically accurate guidance for therapeutic agent delivery. The disclosed system thus represents a significant advance over conventional injection techniques, offering a non-invasive, functionally guided approach to neuromuscular intervention that is applicable across a broad range of clinical conditions, including focal spasticity, cervical and limb dystonia, chronic myofascial pain, and cosmetic applications requiring precise neuromuscular targeting.

Reference will now be made in detail to exemplary aspects of the present disclosure that are illustrated in the accompanying drawings.

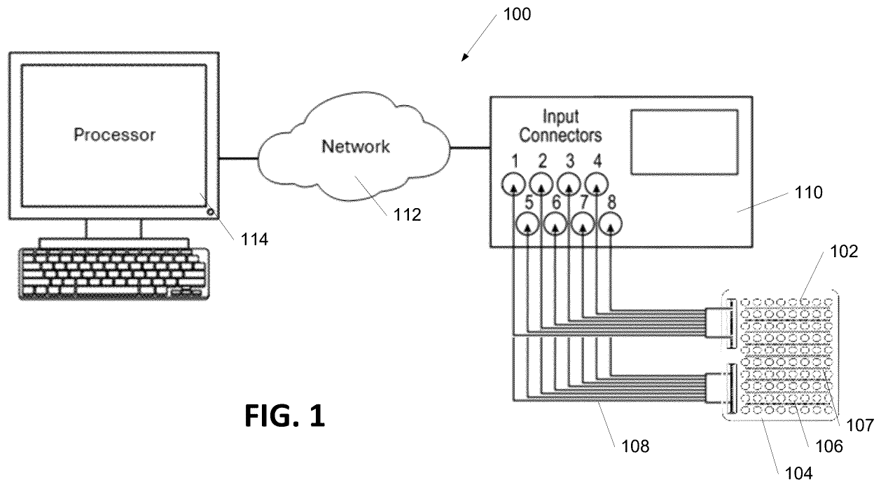

FIG. 1 illustrates an exemplary neuromuscular modulation system 100 configured for both monitoring and therapeutic intervention of neuromuscular activity. The system 100 integrates high-density electromyographic signal acquisition with precision-guided therapeutic delivery, enabling both electrical stimulation therapy and injection-mediated interventions through a unified platform. As shown, the system 100 includes an electrode array 102 that is configured to be positioned adjacent to a subject's body for detecting electromyographic (EMG) signals and facilitating targeted therapeutic interventions at identified neuromuscular regions. The electrode array 102 comprises a flexible substrate 104, which may be fabricated from biocompatible polymeric materials such as thermoplastic urethane (TPU), thermoplastic elastomer (TPE), polyester, or polyimide, thereby allowing the electrode array 102 to conform to body contours and accommodate anatomically varied muscle groups. A plurality of electrodes 106 are disposed on the substrate 104 and may be arranged in various array configurations, such as linear, two-dimensional grid, or three-dimensional arrangements, depending on the muscle group or anatomical region of interest.

In embodiments configured for injection guidance, the flexible substrate 104 further includes a plurality of voids 107 strategically formed within the substrate material. The voids 107 are positioned to facilitate syringe-mediated delivery of neurolytic agents, such as botulinum toxin, directly through the substrate 104 to underlying motor unit territories identified through EMG analysis, while simultaneously preventing contamination of the injection site by substrate material. This integrated design enables clinicians to map motor unit territories based on spatial and temporal characteristics of detected EMG signals and subsequently guide the precise placement and delivery of therapeutic agents to those identified territories without the need to reposition or remove the electrode array 102. The voids 107 may be distributed across the substrate 104 in patterns that align with the spatial sampling characteristics of the electrodes 106, thereby ensuring that injection access is available proximate to regions of identified neuromuscular activity. In certain embodiments, the voids 107 may be prefabricated at regular intervals, while in other embodiments, the void pattern may be customized based on the target muscle architecture or anticipated motor unit distribution.

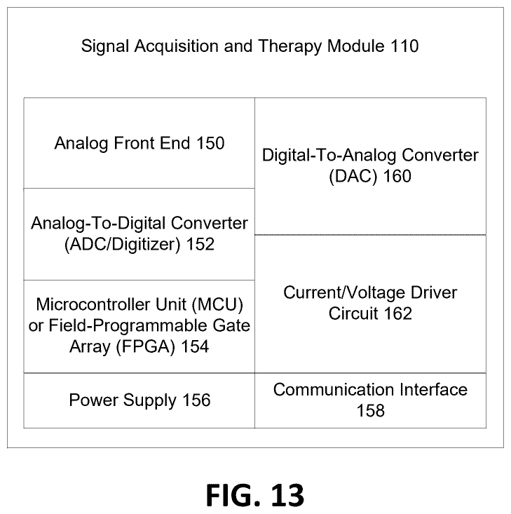

The electrode array 102 is operably connected to a signal acquisition and therapy module 110, which serves as an interface between the electrode array 102 and the computational components of the system 100. The electrode array 102 may be connected to the signal acquisition and therapy module 110 via a wired connection 108, such as a shielded cable with multiple conductor channels corresponding to the plurality of electrodes 106, or alternatively via a wireless connection as described in greater detail below with reference to FIGS. 13-15. In various embodiments, the signal acquisition and therapy module 110 houses analog and digital circuitry for conditioning, amplifying, and digitizing EMG signals obtained from the electrodes 106. The signal acquisition and therapy module 110 may include an analog front end configured to provide high-gain, low-noise amplification of bioelectric signals, an analog-to-digital converter (ADC) for converting conditioned signals into digital data streams, and a microcontroller unit (MCU) or field-programmable gate array (FPGA) for real-time preprocessing and communication management. In embodiments that incorporate electrical stimulation capabilities, the signal acquisition and therapy module 110 further includes waveform generation circuitry, digital-to-analog conversion stages, and current or voltage driver circuits configured to deliver therapeutic stimulation waveforms to selected electrodes 106 within the array. The signal acquisition and therapy module 110 may also include a power supply, such as a rechargeable battery pack, and control circuitry to coordinate data acquisition, therapy delivery, and bidirectional communication with external computing devices.

The signal acquisition and therapy module 110 communicates with a processor 114 through a network 112. The network 112 may be implemented as a wired communication link, such as USB, Ethernet, or a dedicated data cable connection, or as a wireless communication link, such as Bluetooth®, Wi-Fi®, Zigbee®, or another short-range or long-range wireless protocol. The processor 114 may be implemented in a computing device, such as a laptop computer, tablet computer, desktop workstation, or dedicated embedded processing unit, and is configured to execute software instructions for advanced signal processing, pattern recognition, and therapeutic decision-making. Specifically, the processor 114 processes EMG data received from the signal acquisition and therapy module 110 to identify neuromuscular junction regions within target muscles, determine whether a muscle is in a hypertonic state (characterized by excessive resting activity, spasticity, or involuntary contraction) or a hypotonic state (characterized by weakness, atrophy, or insufficient motor unit recruitment), and select an appropriate therapeutic strategy based on the physiological state of the identified neuromuscular regions. For injection-guided applications, the processor 114 generates spatial maps, such as root mean square (RMS) heatmaps or innervation zone point clouds, that guide clinicians in selecting optimal injection coordinates corresponding to voids 107 within the electrode array 102. For electrical stimulation applications, the processor 114 determines stimulation parameters such as frequency, amplitude, pulse width, and duty cycle, and communicates these parameters back to the signal acquisition and therapy module 110 for delivery through selected electrodes 106.

In this manner, the system 100 enables closed-loop neuromuscular modulation in which EMG signals are acquired by the electrode array 102, transmitted to the signal acquisition and therapy module 110, processed and analyzed by the processor 114, and therapeutic interventions—whether electrical stimulation or injection guidance—are delivered back through the electrode array 102 under coordinated control of the processor 114 and the signal acquisition and therapy module 110. Such integration allows for precision targeting of neuromuscular junction territories for muscle strengthening, muscle relaxation, and injection-mediated therapies such as botulinum toxin delivery, thereby overcoming the limitations of traditional blind injection techniques and conventional electrical stimulation approaches. The system 100 thus provides a comprehensive, functionally guided platform for addressing a wide range of neuromuscular disorders, including spasticity, dystonia, chronic pain syndromes, pelvic floor dysfunction, and focal muscle hypertonicity, with applications spanning clinical, rehabilitative, and aesthetic treatment contexts.

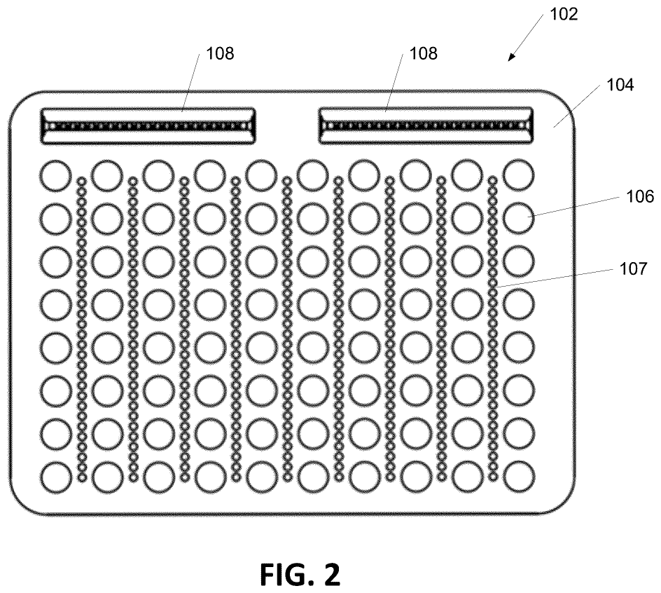

FIGS. 2-6 illustrate various views of the flexible electrode array 102, which is configured to be aligned with underlying muscle fibers in a parallel orientation to optimize the detection of electromyographic signals and facilitate targeted therapeutic delivery. As shown in the plan view of FIG. 2, the electrode array 102 comprises a flexible substrate 104 fabricated from a biocompatible polymeric material selected from the group consisting of thermoplastic urethane (TPU), thermoplastic elastomer (TPE), polyester, and polyimide. The flexible substrate 104 provides structural support for the electrode array 102 while allowing the array to conform to varied anatomical contours, including curved surfaces such as limbs, the torso, and other body regions. The flexibility of the substrate 104 ensures that the electrode array 102 maintains intimate contact with the skin surface during muscle activation, thereby reducing motion artifacts and improving signal fidelity. In embodiments configured for injection guidance, the flexible substrate 104 is sufficiently compliant to accommodate needle penetration through the voids 107 without tearing, cracking, or deforming in a manner that would compromise the integrity of the electrode array 102 or the sterility of the injection site.

The electrode array 102 includes a plurality of conductive traces deposited on the flexible substrate 104. The conductive traces may be formed using thin-film deposition techniques, such as sputtering, chemical vapor deposition, or inkjet printing, and may be patterned using photolithographic methods to create precise geometric layouts. The conductive traces are fabricated from one or more electrically conductive materials selected from the group consisting of copper, gold, platinum, and silver, each chosen for its electrical conductivity, biocompatibility, and resistance to corrosion. In certain embodiments, the conductive traces may include a layered structure, such as a copper base layer for bulk conductivity and a gold or platinum surface layer for enhanced biocompatibility and durability. The conductive traces extend from a connection interface at one end of the substrate 104 to individual contact pads 106 distributed across the surface of the array. As shown in FIG. 2, the conductive traces may be arranged in parallel or radial patterns depending on the muscle architecture and the intended spatial sampling resolution.

A plurality of electrodes 106, also referred to as contact pads, are operably connected to the conductive traces and are exposed on the skin-facing surface of the flexible substrate 104 to enable direct electrical contact with the subject's skin. Each contact pad 106 is constructed of a conductive material selected from the group consisting of gold, silver, silver/silver chloride (Ag/AgCl), stainless steel, and platinum/iridium (Pt/Ir), materials known for their low contact impedance, electrochemical stability, and compatibility with bioelectric signal acquisition. The contact pads 106 are spatially distributed on the substrate 104 at intervals corresponding to a sampling frequency less than or equal to the Nyquist frequency of the target muscle signals, thereby ensuring that the electrode array 102 can accurately capture the spatial distribution of motor unit action potentials without spatial aliasing. In various embodiments, the contact pads 106 may have circular, rectangular, or polygonal geometries, with diameters or characteristic dimensions ranging from approximately 2 mm to 10 mm, depending on the muscle group being monitored and the desired spatial resolution. The arrangement of the contact pads 106 may be linear, as shown in FIG. 2, for parallel-fibered muscles such as the biceps brachii, or may be configured in two-dimensional grids or three-dimensional arrays for pennate or multipennate muscle architectures.

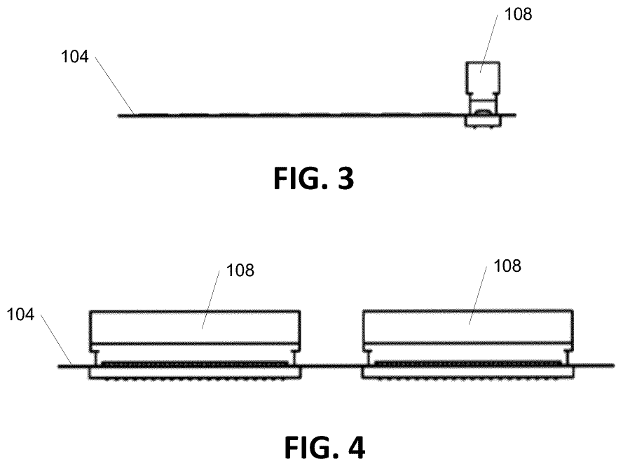

As illustrated in the side profile views of FIGS. 3 and 4, the flexible substrate 104 has a layered construction that includes the conductive traces embedded or deposited within or on the substrate material, with the contact pads 106 extending through or positioned on the outer surface to make contact with the skin. The substrate 104 may have a thickness ranging from approximately 50 micrometers to 500 micrometers, balancing mechanical flexibility with structural integrity. In certain embodiments, the substrate 104 may include multiple layers, such as a base layer providing mechanical support, an intermediate layer containing the conductive traces, and a top layer providing electrical insulation and protection for the traces while exposing the contact pads 106. The side profile views further illustrate the low-profile design of the electrode array 102, which minimizes bulk and allows the array to be worn comfortably under clothing or secured to the skin using adhesive, straps, or medical tape.

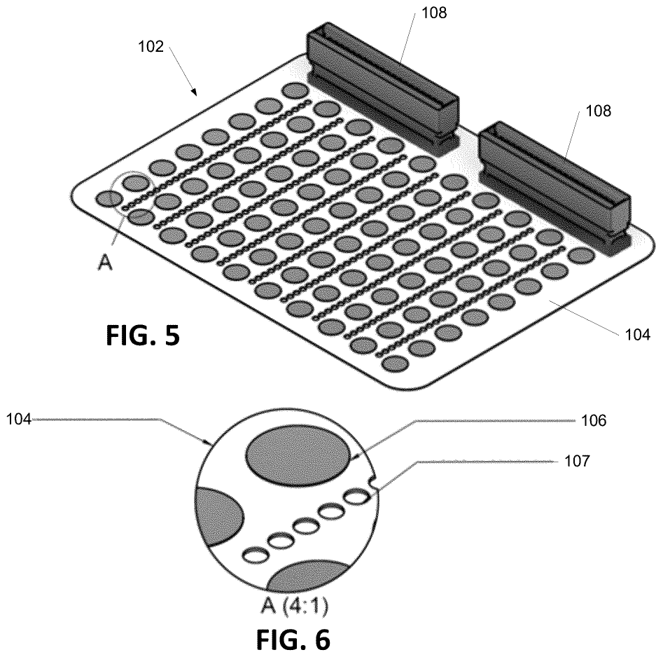

The perspective view shown in FIG. 5 provides a three-dimensional representation of the electrode array 102, illustrating the spatial relationship between the flexible substrate 104, the conductive traces, and the contact pads 106. This view emphasizes the conformability of the electrode array 102, showing how the substrate 104 can bend and flex to accommodate anatomical curvature while maintaining electrical connectivity between the contact pads 106 and the signal acquisition and therapy module 110 via the wired connection 108. The perspective view further illustrates the integration of the electrode array 102 into a unified, compact device suitable for clinical and home-based applications.

FIG. 6 provides a close-up view of the flexible electrode array 102, highlighting the critical structural features that enable the array to function as both a signal acquisition device and an injection guidance platform. As shown, the flexible substrate 104 includes a plurality of voids 107 strategically formed within the substrate material. The voids 107 are apertures, openings, or through-holes that extend completely through the thickness of the substrate 104, thereby providing unobstructed access for syringe needles or other injection implements to penetrate the substrate and deliver therapeutic agents, such as neurolytic agents including botulinum toxin, directly to underlying motor unit territories. The voids 107 are positioned to correspond spatially with the contact pads 106, such that regions of high neuromuscular activity identified through electromyographic mapping can be accessed for injection without the need to reposition or remove the electrode array 102. In various embodiments, the voids 107 may have circular, elliptical, or polygonal cross-sections, with diameters ranging from approximately 1 mm to 5 mm, dimensioned to accommodate standard hypodermic needles while minimizing the loss of structural integrity of the substrate 104. The voids 107 are further configured to inhibit contamination of the injection site by preventing substrate material from being displaced or introduced into the subject's body during syringe-mediated transfer of the therapeutic agent. By integrating the voids 107 directly into the flexible substrate 104, the electrode array 102 enables a seamless workflow in which electromyographic mapping, motor unit territory identification, and targeted injection can be performed sequentially using a single device, thereby improving procedural efficiency, precision, and clinical outcomes.

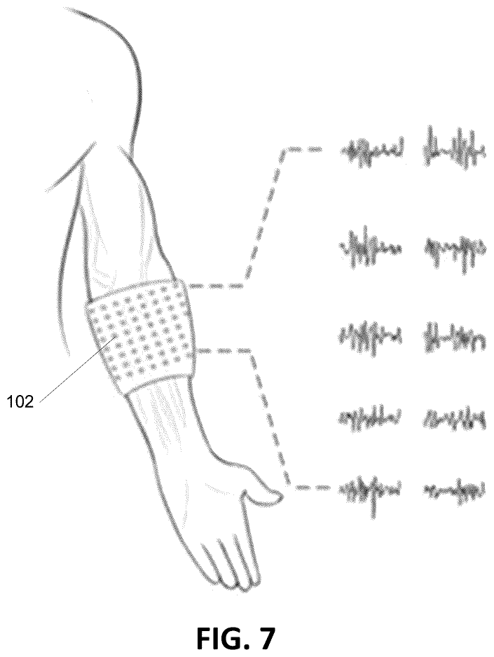

FIG. 7 illustrates an exemplary configuration in which the electrode array 102 is positioned externally on the arm of a patient to detect muscle activity and deliver electrical stimulation or to guide therapeutic injection. In the depicted embodiment, the electrode array 102 is applied to the skin surface overlying a target muscle group, such as the biceps brachii, triceps, or forearm flexor muscles, to facilitate both recording of electromyographic (EMG) signals and subsequent therapeutic modulation. The electrode array 102 comprises the flexible substrate 104 with the plurality of electrodes 106 distributed thereon, and is operably connected to the signal acquisition and therapy module 110 via the wired connection 108. The flexible substrate 104 enables the electrode array 102 to conform to the contours of the arm and maintain intimate contact with the skin surface during dynamic activities, such as when the patient performs predefined movement protocols intended to activate specific muscle groups.

In one embodiment, the flexible substrate 104 of the electrode array 102 is configured to resemble a strip or patch having characteristics similar to medical-grade adhesive tape or an adhesive bandage. The substrate 104 includes a skin-facing adhesive side that permits secure placement of the electrode array 102 against the patient's skin during the diagnostic or therapeutic procedure. The adhesive side may be coated with a biocompatible adhesive formulation, such as a pressure-sensitive acrylic adhesive, hydrocolloid adhesive, or silicone-based adhesive, that provides sufficient tack to maintain reliable electrode-to-skin contact without causing irritation, discomfort, or damage to the patient's skin upon removal. The adhesive properties of the substrate 104 are selected such that the electrode array 102 may be applied and removed multiple times without significant degradation of adhesive performance or signal quality, thereby enabling repeated use in clinical or at-home settings.

This tape-like or patch-like configuration enables the electrode array 102 to remain securely fixed in place during dynamic activities, preventing unwanted electrode displacement, motion artifacts, or signal degradation that could compromise the fidelity of recorded EMG signals or the accuracy of therapeutic delivery. Secure positioning is particularly important when the patient performs voluntary muscle contractions, resistance exercises, or spasticity-inducing protocols, as such activities can generate substantial skin movement and electrode-tissue interface perturbations. The adhesive attachment ensures that the electrodes 106 maintain consistent electrical contact with the skin throughout the measurement and therapy session, thereby improving both signal quality and therapeutic precision.

As further illustrated in FIG. 7, the flexible substrate 104 may include the plurality of voids 107 strategically formed within the substrate material to facilitate syringe-mediated delivery of therapeutic agents. In embodiments configured for injection guidance, the voids 107 provide unobstructed access for hypodermic needles to penetrate the substrate 104 and deliver neurolytic agents, such as botulinum toxin, directly to underlying motor unit territories identified through EMG analysis. The voids 107 are positioned to correspond spatially with the electrodes 106, such that regions of high neuromuscular activity identified through electromyographic mapping can be accessed for injection without the need to reposition or remove the electrode array 102 from the patient's arm. This integrated design enables a seamless clinical workflow in which EMG signal acquisition, motor unit territory mapping, and targeted injection can be performed sequentially using a single device, thereby improving procedural efficiency and reducing the risk of targeting errors that can occur when electrode arrays are repositioned between diagnostic and therapeutic phases.

At the conclusion of a therapeutic session, the electrode array 102 may be removed or peeled away from the patient's skin by gently lifting an edge of the flexible substrate 104 and progressively detaching the adhesive surface from the skin. In some embodiments, the adhesive side of the substrate 104 may be designed as a single-use disposable surface to ensure sterility and hygiene, particularly in clinical applications involving injection of therapeutic agents. In other embodiments, the adhesive layer may be replaceable or reconditionable for repeated use, such as by applying a fresh adhesive overlay or cleaning and reactivating the existing adhesive surface. This design facilitates clinical workflows in hospital, outpatient, and rehabilitation settings, while also supporting potential at-home use by patients requiring repeated therapeutic sessions for chronic neuromuscular conditions, such as spasticity management, muscle strengthening following stroke or injury, or ongoing pain modulation therapy.

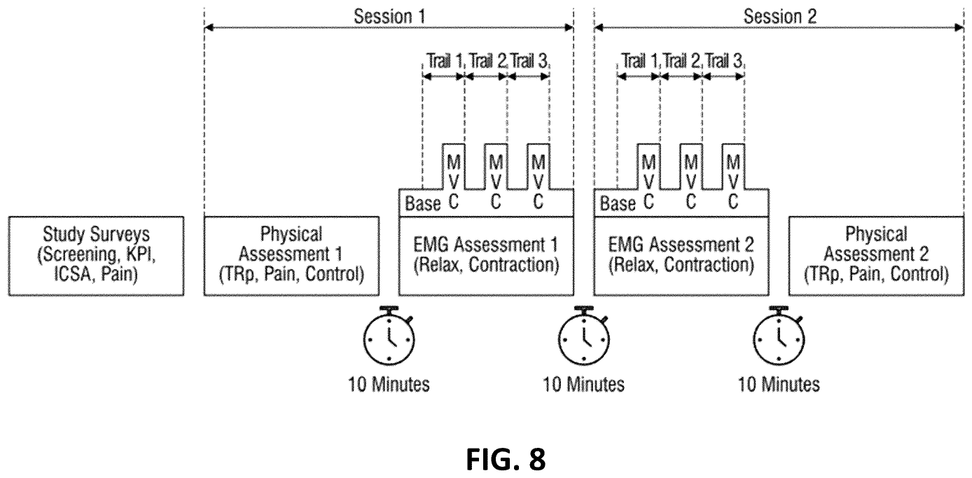

FIG. 8 illustrates an exemplary testing protocol for evaluating neuromuscular activity and assessing treatment efficacy using the neuromuscular modulation system 100. The protocol is organized into a series of sequential assessment blocks with intervening rest periods to standardize the acquisition of electromyographic (EMG) data and clinical examination results. The testing protocol is designed to establish baseline neuromuscular function, monitor changes in muscle activity during therapeutic intervention, and provide objective quantitative metrics for assessing treatment response. The protocol depicted in FIG. 8 is particularly well-suited for evaluating conditions involving altered muscle tone, such as spasticity, dystonia, chronic myofascial pain, pelvic floor dysfunction, or muscle weakness following injury or neurological impairment, though it may be adapted to other clinical applications as needed.

The protocol begins with a preliminary assessment phase that includes study surveys and screening measures. In this phase, baseline symptomatology and patient-reported outcomes are collected prior to the commencement of instrumented measurements. Examples of preliminary assessments may include screening questionnaires, key performance indicators (KPIs), symptom-specific assessments (such as interstitial cystitis symptom assessment or pelvic pain indices), visual analog pain scales, or functional status evaluations. These preliminary measures establish a baseline clinical profile against which subsequent changes can be compared and provide context for interpreting the electromyographic findings obtained during the protocol.

Following the preliminary assessments, the protocol enters Session 1, which is indicated to last approximately 10 minutes. Session 1 comprises two sequential assessment blocks: Physical Assessment 1 and EMG Assessment 1. During Physical Assessment 1, the clinician performs a manual examination to identify trigger points (TRp), assess pain levels, and evaluate motor control or functional status. Trigger points may be identified through palpation, with pain levels scored using standardized scales such as a numeric rating scale (NRS) or visual analog scale (VAS). Motor control evaluation may include assessments of voluntary contraction strength, coordination, endurance, or the presence of involuntary contractions or spasms. This physical assessment provides clinically relevant contextual information that complements the objective electrophysiological data obtained during EMG Assessment 1.

EMG Assessment 1 follows the physical assessment and involves the use of the electrode array 102 and associated system components to record high-density surface EMG (HD-sEMG) signals during both resting and contraction phases. In the embodiment shown, EMG Assessment 1 begins with a baseline maximum voluntary contraction (MVC) measurement to establish the patient's maximal muscle activation capacity. Following the baseline MVC, the protocol proceeds through a series of alternating resting trials and MVC trials, specifically Trial 1, Trial 2, and Trial 3. During each resting trial, the patient is instructed to remain as relaxed as possible while EMG signals are recorded to capture baseline muscle activity, which may be elevated in conditions involving spasticity, hypertonicity, or involuntary muscle activation. During each MVC trial, the patient is instructed to perform a maximal voluntary contraction of the target muscle group, enabling the system to capture peak activation levels and assess motor unit recruitment patterns. The alternating structure of resting and MVC trials enables the system to compute metrics such as resting-to-MVC ratios, which provide quantitative indicators of neuromuscular dysfunction, including hypertonicity indices or muscle weakness indices.

At the conclusion of Session 1, the patient is provided with a rest period of approximately 10 minutes. This break serves multiple purposes: it reduces muscular fatigue that could confound subsequent measurements, allows metabolic recovery of the muscle tissue, normalizes muscle activity levels following the exertion of MVC trials, and provides the patient with an opportunity to rest and reposition if needed. The rest period is a critical component of the protocol, as it ensures that Session 2 measurements are not artificially influenced by residual fatigue or altered neuromuscular state from Session 1. In clinical practice, the rest period may also be used to review preliminary results, adjust electrode positioning if necessary, or provide the patient with feedback regarding their performance during Session 1.

Following the rest period, the protocol enters Session 2, which again lasts approximately 10 minutes and mirrors the structure of Session 1. Session 2 begins with Physical Assessment 2, during which the clinician repeats the trigger point identification, pain scoring, and motor control evaluation performed in Physical Assessment 1. Repeating the physical assessment allows the clinician to detect any immediate changes in muscle tone, pain levels, or functional status that may have occurred during or following Session 1, and provides a basis for assessing intra-session variability or responsiveness to therapeutic interventions. Physical Assessment 2 is followed by EMG Assessment 2, which likewise includes a baseline MVC measurement and alternating resting and MVC trials (Trial 1, Trial 2, Trial 3). The structure and procedures of EMG Assessment 2 are identical to those of EMG Assessment 1, enabling direct comparison of electromyographic data between the two sessions and supporting statistical analysis of repeatability, consistency, and treatment-induced changes.

The HD-sEMG data collected across both Session 1 and Session 2 may be processed by the processor 114 to extract clinically meaningful metrics and generate spatial maps of neuromuscular activity. For example, the resting EMG data and MVC EMG data may be used to calculate a pelvic muscle hypertonicity index or other condition-specific indices. In one embodiment, the hypertonicity index may be defined as a resting root mean square (RMS) ratio derived from the HD-sEMG signals, computed by normalizing the RMS values obtained during resting trials against the RMS values obtained during MVC trials. The calculated index provides an objective, quantitative measure of muscle tone, enabling classification of muscle activity as hypertonic (elevated resting activity relative to voluntary activation), hypotonic (reduced voluntary activation capacity), or normal. The data collected through the FIG. 8 protocol may further be used to monitor treatment response over time by repeating the protocol at intervals following therapeutic intervention, refine therapeutic strategies based on observed changes in neuromuscular metrics, and provide standardized endpoints for clinical evaluation, research studies, or regulatory submissions. By establishing a repeatable and standardized testing framework, the protocol depicted in FIG. 8 enhances the clinical utility of the neuromuscular modulation system 100 and supports evidence-based decision-making in the management of neuromuscular disorders.

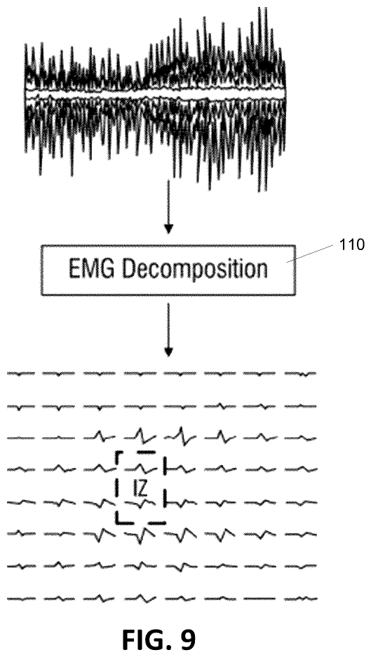

FIG. 9 illustrates an exemplary process of EMG decomposition as performed by the neuromuscular modulation system 100. The decomposition process transforms high-density surface electromyographic (HD-sEMG) signals acquired from the electrode array 102 into spatially and temporally resolved motor unit action potentials (MUAPs), enabling the system to identify discrete motor unit territories and innervation zones within target muscles. This decomposition is a critical component of the system's ability to provide precision-guided therapeutic delivery, as it allows the system to isolate individual motor units and determine their anatomical locations, thereby enabling targeted electrical stimulation or injection guidance to specific neuromuscular junction regions.

During operation, HD-sEMG signals are recorded from the electrode array 102 while a patient performs a set of predefined movement protocols designed to activate specific muscles or motor unit territories. The signals may first be preprocessed using band-pass filtering between approximately 10 Hz and 500 Hz, for example using a second-order Butterworth filter, to isolate relevant electromyographic frequencies while attenuating out-of-band noise. In addition, a 60 Hz notch filter, such as a Butterworth notch filter, may be applied to attenuate mains interference commonly present in clinical and laboratory environments. The filtered signals are then segmented into trials according to the experimental paradigm, which may include alternating resting and maximum voluntary contraction (MVC) phases as described with reference to FIG. 8.3

Root mean square (RMS) values are calculated channel-wise in half-second intervals for each resting trial and averaged across trials. A resting RMS ratio may be determined for each channel by normalizing the averaged resting RMS values against the peak RMS values observed during the corresponding MVC trial. These ratios may be averaged across multiple resting trials in each session to generate a high-resolution spatial map of muscle activity, such as a 64-channel resting RMS ratio heatmap. This map highlights regions of altered neuromuscular activity and serves as an input for further decomposition and motor unit identification. Regions exhibiting elevated resting activity relative to MVC activity may indicate hypertonicity, spasticity, or involuntary muscle activation, while regions exhibiting reduced MVC activity may indicate weakness, atrophy, or impaired motor unit recruitment.

As depicted in FIG. 9, the filtered HD-sEMG signals acquired during resting and contraction phases are decomposed into motor unit action potentials (MUAPs) using advanced signal processing algorithms. In one embodiment, a k-means clustering convolution kernel compensation algorithm is employed to identify and separate overlapping motor unit signals. Each MUAP represents the summated electrical potential of muscle fibers innervated by a single motor neuron and activated during voluntary contraction. By identifying the MUAPs across the multiple channels of the electrode array 102, the system can characterize the functional contributions of discrete motor units to the overall muscle activity and determine the spatial distribution of motor unit territories within the monitored muscle group.

The innervation zone (IZ) of each motor unit, corresponding to the neuromuscular junction region where motor nerve terminals synapse with muscle fibers, may be identified from a bipolar map of the MUAPs. The IZ is detected by analyzing the phase reversal of the propagating action potential signals along the muscle fibers, which signifies the anatomical location where action potentials are initiated. By overlaying these IZ determinations across all channels of the electrode array 102, the system constructs a spatially resolved map of neuromuscular junction territories within the monitored muscle group. This map provides precise anatomical coordinates for subsequent therapeutic interventions, whether electrical stimulation for muscle modulation or injection guidance for neurolytic agent delivery.

Advanced signal processing techniques may be further applied to refine the decomposition results and enhance the accuracy of motor unit localization. For example, the system may employ blind source separation algorithms, such as independent component analysis (ICA) or FastICA, to unmix overlapping signals and isolate individual motor unit contributions from the composite EMG recordings. The system may compute projections of weight matrices derived from the decomposition and cluster recording sites based on statistical correlation, thereby grouping electrodes that detect activity from the same motor unit. Spatial filter banks may then be constructed to isolate activity specific to individual motor unit territories. These filter banks function as beamforming algorithms that enhance signals originating from specific spatial locations while attenuating signals from other regions, thereby providing targeted identification of activation zones and enabling precise anatomical coordinates for delivering electrical stimulation or guiding injection needles through the voids 107 in the flexible substrate 104.

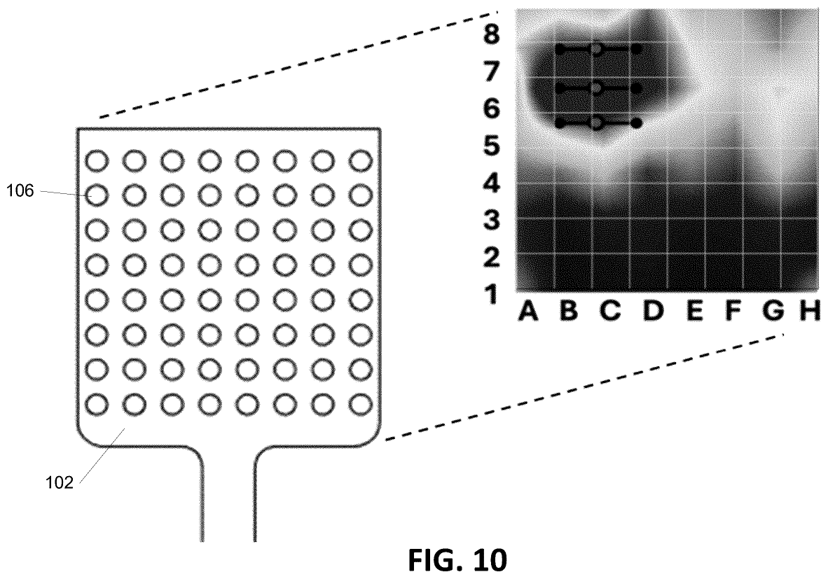

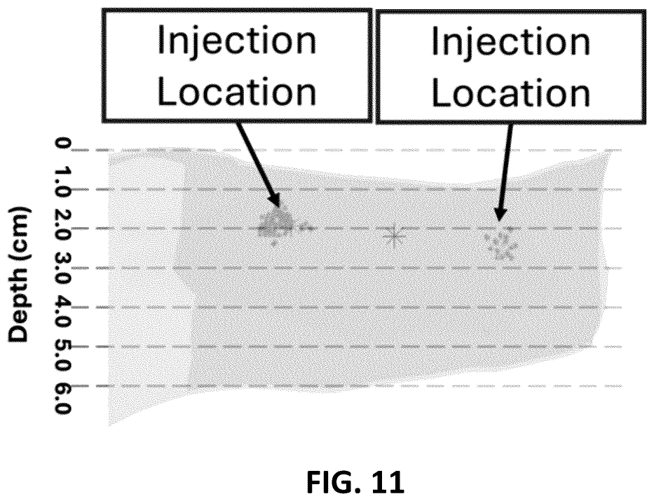

The output of the EMG decomposition process depicted in FIG. 9 includes spatially resolved maps of motor unit territories, quantitative metrics such as motor unit firing rates and recruitment thresholds, and anatomical coordinates for identified innervation zones. These outputs are transmitted to the processor 114, which uses the decomposition results to determine the appropriate therapeutic strategy. For hypertonic motor unit territories exhibiting excessive resting activity, the processor 114 may select high-frequency blocking stimulation to induce muscle relaxation, as illustrated in FIGS. 10-11. For hypotonic motor unit territories exhibiting weak voluntary activation, the processor 114 may select modulated high-frequency stimulation with a low-frequency envelope to promote muscle strengthening, as illustrated in FIGS. 10-11. By integrating EMG decomposition with adaptive therapy selection, the system 100 provides closed-loop neuromuscular modulation that is personalized to the physiological state of the target muscle group, thereby maximizing therapeutic efficacy and clinical outcomes.

FIG. 10 is an illustration depicting an exemplary location root mean square (RMS) heatmap of the motor endplate territories generated from high-density EMG signal analysis highlighting regions of high neuromuscular activity intensity for targeted therapeutic delivery. The heatmap is a visual representation produced by the processor 114 following the EMG decomposition process described with reference to FIG. 9, and provides clinicians with spatially resolved guidance for selecting optimal sites for either electrical stimulation or injection-mediated therapeutic interventions. In the depicted embodiment, the heatmap is organized as a grid corresponding to the spatial arrangement of the electrodes 106 within the electrode array 102, with each grid position representing the electromyographic activity detected by a corresponding electrode or region of electrodes during the testing protocol.

The RMS heatmap is generated by calculating root mean square values of the EMG signals recorded from each electrode 106 during resting trials and normalizing these values against the peak RMS values observed during maximum voluntary contraction (MVC) trials, as described with reference to FIGS. 8 and 9. The resulting resting RMS ratio for each electrode provides a quantitative metric of neuromuscular activity at that spatial location, with elevated ratios indicating regions of high resting activity characteristic of hypertonicity, spasticity, or involuntary muscle activation, and reduced ratios indicating regions of weak voluntary activation characteristic of hypotonicity, muscle weakness, or atrophy. The calculated RMS ratios are then mapped onto a color-coded or intensity-scaled visualization, such that regions of heightened activity appear as “hot spots” on the heatmap, while regions of reduced activity appear as “cold spots.” This visual representation enables clinicians to rapidly identify motor endplate territories requiring therapeutic intervention without the need to manually interpret raw numerical data from dozens of individual electrode channels.

In the embodiment shown in FIG. 10, the heatmap identifies one or more discrete regions of elevated neuromuscular activity, corresponding to motor endplate territories exhibiting pathological activation patterns. These identified regions are indicated on the heatmap as zones of high intensity, which may be displayed using color gradients (e.g., red for high activity, blue for low activity), grayscale intensity levels, or numerical overlays providing specific RMS ratio values. The spatial resolution of the heatmap is determined by the density and arrangement of the electrodes 106 within the electrode array 102, with higher electrode densities providing finer spatial discrimination of motor unit territories. In various embodiments, the heatmap may represent activity from a linear electrode array aligned with parallel-fibered muscles, a two-dimensional grid array positioned over complex muscle groups, or a three-dimensional array configuration adapted to pennate or multipennate muscle architectures.

For applications involving injection guidance, the RMS heatmap serves as a targeting map to direct the clinician in selecting appropriate voids 107 within the flexible substrate 104 through which to deliver neurolytic agents, such as botulinum toxin. As illustrated in FIG. 10, specific injection locations may be identified and marked on the heatmap, indicating the coordinates of voids 107 that are spatially aligned with the identified motor endplate territories exhibiting elevated activity. By delivering the neurolytic agent through voids 107 positioned over high-activity zones, the system ensures that the therapeutic agent is deposited directly at or near the motor unit territories responsible for pathological muscle activity, thereby maximizing treatment efficacy while minimizing the dosage required and reducing the risk of off-target effects on adjacent healthy muscle tissue. The heatmap thus provides a functionally guided, objective basis for injection site selection, replacing subjective palpation-based or anatomical landmark-based methods that lack the spatial and functional resolution necessary for optimal neurolytic agent delivery.

For applications involving electrical stimulation therapy, the RMS heatmap guides the processor 114 in selecting specific electrodes 106 through which to deliver therapeutic stimulation waveforms. When the heatmap identifies regions of hypertonicity characterized by excessive resting activity, the processor 114 may direct the signal acquisition and therapy module 110 to deliver high-frequency blocking stimulation (approximately 1 kHz to 10 kHz) through electrodes 106 aligned with those hypertonic motor endplate territories, as described in greater detail with reference to FIGS. 10-11. Conversely, when the heatmap identifies regions of hypotonicity characterized by weak voluntary activation, the processor 114 may direct the delivery of modulated high-frequency stimulation with a low-frequency envelope (approximately 10 Hz to 50 Hz modulation) to promote muscle strengthening. By integrating the spatial information provided by the RMS heatmap with adaptive therapy selection logic, the system 100 enables personalized, closed-loop neuromuscular modulation that is responsive to the specific physiological state of the target muscle group, thereby addressing both muscle overactivity and muscle weakness within a unified therapeutic platform.

FIG. 11 illustrates innervation zone (IZ) point clouds, further refining the mapping shown in FIG. 10 by demonstrating a depth and spatial distribution of identified IZ regions to guide precise injection coordinates. The point cloud representation provides a three-dimensional anatomical map of neuromuscular junction territories, extending beyond the two-dimensional surface mapping of the RMS heatmap to include depth information that is critical for accurate needle penetration and therapeutic agent delivery. By determining not only the lateral position but also the estimated depth of innervation zones within the muscle tissue, the system 100 enables clinicians to select both the appropriate void 107 for needle insertion and the optimal insertion angle and depth for reaching the target motor unit territory.

The innervation zone point clouds depicted in FIG. 11 are generated by applying bioelectric source imaging techniques to the high-density EMG signals acquired by the electrode array 102. As described with reference to FIG. 9, the system processes the EMG signals using motor unit decomposition algorithms, blind source separation techniques, and spatial filtering to identify discrete motor unit action potentials (MUAPs) and their corresponding innervation zones. The innervation zones represent the neuromuscular junction regions where motor nerve terminals synapse with muscle fibers, and are detected by analyzing the phase reversal characteristics of propagating action potentials along the muscle fiber direction. By examining the spatial distribution of these phase reversals across the multiple channels of the electrode array 102, the system constructs a spatially resolved map of IZ locations.

To determine the depth of each identified innervation zone, the system employs inverse problem-solving techniques based on the attenuation and spatial spreading of bioelectric potentials as they propagate from the source (the neuromuscular junction) through muscle tissue to the recording electrodes at the skin surface. In one embodiment, spatial filter banks are constructed based on the identified motor unit territories, and the amplitude distribution and decay characteristics of the signals across adjacent electrodes are analyzed to estimate the distance of the bioelectric source from the electrode array 102. Sources located deeper within the muscle tissue exhibit broader spatial distributions and lower amplitude recordings compared to sources located superficially, thereby enabling depth estimation through analysis of the spatial extent and magnitude of the recorded signals. In various embodiments, the depth estimation may further incorporate tissue impedance models, muscle fiber orientation data, and anatomical constraints derived from the known geometry of the target muscle group.

The resulting innervation zone point cloud, as illustrated in FIG. 11, displays each identified IZ as a discrete point in three-dimensional space, with coordinates corresponding to the lateral position on the muscle surface (e.g., x and y coordinates aligned with the grid structure of the electrode array 102) and a depth coordinate (e.g., z-axis) representing the estimated distance of the IZ below the skin surface. In the depicted embodiment, depth values may range from approximately 0 cm (at the skin surface) to 6 cm or more, depending on the muscle group and the penetration depth of the bioelectric source imaging analysis. Multiple IZ points corresponding to different motor units within the target muscle are displayed simultaneously, providing a comprehensive anatomical map of all identifiable neuromuscular junction regions within the field of view of the electrode array 102.

For applications involving injection-guided therapeutic delivery, the innervation zone point clouds of FIG. 11 provide precise injection coordinates that specify not only the surface location for needle insertion (identified by the corresponding void 107 in the flexible substrate 104) but also the recommended insertion depth and trajectory for reaching the target motor unit territory. In one embodiment, the processor 114 may calculate and display the optimal needle insertion angle based on the lateral offset between the selected void 107 and the target IZ location, combined with the estimated depth of the IZ. This guidance ensures that the neurolytic agent, such as botulinum toxin, is deposited directly at or near the neuromuscular junction where motor nerve activity is most concentrated, thereby maximizing therapeutic efficacy while minimizing the total dosage required and reducing the risk of off-target effects on adjacent muscle groups.

The integration of depth information further enhances the precision of therapeutic delivery in anatomically complex or deeply situated musculature, where surface landmarks and conventional imaging modalities provide limited guidance. For example, in the treatment of deep spasticity affecting the gastrocnemius muscle or other pennate muscles, the innervation zone point clouds enable clinicians to target specific motor unit territories at varying depths within the muscle architecture, accounting for the oblique orientation of muscle fibers and the three-dimensional distribution of motor endplate regions. Similarly, in the treatment of pelvic floor dysfunction, where internal muscles are accessed via insertable tools as described with reference to FIGS. 4-5, the depth and spatial distribution information provided by the point clouds enables precise delivery of therapeutic agents to internal motor unit territories that are otherwise difficult to visualize or localize using conventional techniques.

The processor 114 may further refine the innervation zone point clouds by overlaying the IZ locations onto anatomical reference images, such as ultrasound images, MRI scans, or standardized anatomical models corresponding to the target muscle group. This overlay provides clinicians with additional spatial context, enabling correlation of the functionally identified motor unit territories (derived from EMG analysis) with anatomical structures visible through imaging modalities. In certain embodiments, the system may generate injection planning reports that include the point cloud visualization, recommended injection coordinates, estimated insertion depths, and procedural notes, thereby supporting standardized documentation and facilitating communication among clinical team members.

By providing both surface-level activity mapping (FIG. 10) and depth-resolved anatomical coordinates (FIG. 11), the system 100 enables a comprehensive, multi-dimensional approach to neuromuscular junction targeting that significantly advances beyond the capabilities of conventional blind injection or ultrasound-guided techniques. The combination of high-density surface EMG acquisition, advanced signal processing, bioelectric source imaging, and integrated injection guidance transforms neurolytic agent delivery from an anatomically approximate procedure to a functionally precise, data-driven therapeutic intervention with enhanced clinical outcomes.

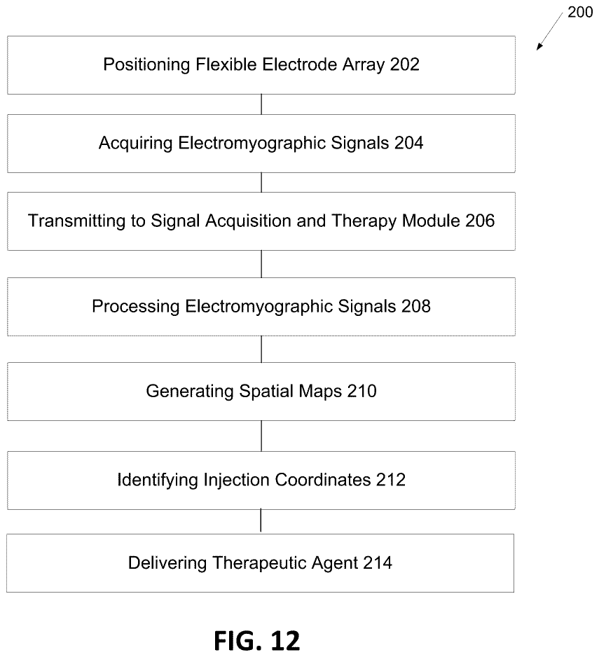

FIG. 12 is a flowchart depicting a method 200 of using a system for electromyography (EMG)-guided injection via a flexible electrode array. The method 200 provides a systematic workflow for precision-guided delivery of therapeutic agents, such as neurolytic agents including botulinum toxin, directly to motor unit territories identified through electromyographic analysis. By integrating high-density surface EMG acquisition with spatial mapping and injection guidance, the method 200 enables clinicians to overcome the limitations of conventional blind injection or ultrasound-guided techniques, which lack the functional resolution necessary to target discrete motor endplate regions. The method 200 is particularly well-suited for the treatment of neuromuscular disorders characterized by focal hyperactivity, spasticity, dystonia, or chronic pain syndromes, and may be applied to a wide range of anatomical regions including upper and lower limb musculature, cervical muscles, facial muscles, and pelvic floor muscles.

The method 200 begins at step 202, in which a flexible electrode array 102 is positioned on a skin surface overlying a target muscle. The flexible electrode array 102 comprises a flexible substrate 104 fabricated from a biocompatible polymeric material, such as thermoplastic urethane (TPU), thermoplastic elastomer (TPE), polyester, or polyimide, which allows the array to conform to anatomical contours and maintain intimate contact with the skin surface throughout the procedure. The electrode array 102 includes a plurality of electrodes 106 spatially distributed on the substrate 104 at intervals corresponding to a sampling frequency less than or equal to the Nyquist frequency of the target muscle signals, thereby ensuring accurate spatial resolution of motor unit activity. The substrate 104 further includes a plurality of voids 107 formed within the substrate material, which are strategically positioned to facilitate syringe-mediated delivery of therapeutic agents while preventing contamination of the injection site by substrate material. The electrode array 102 is aligned with the target muscle such that the orientation of the electrodes 106 corresponds to the muscle fiber direction, which may be parallel for parallel-fibered muscles such as the biceps brachii or may follow the pennation angle for pennate or multipennate muscles such as the gastrocnemius. In certain embodiments, the substrate 104 includes an adhesive backing that secures the electrode array 102 to the skin surface, thereby preventing motion artifacts and ensuring consistent electrode-to-skin contact during dynamic muscle activation protocols.

At step 204, electromyographic signals are acquired from the electrode array 102 during muscle activation. The patient is instructed to perform a predefined series of movements designed to activate the target musculature, thereby eliciting electromyographic activity that can be recorded and analyzed to identify motor unit territories. For upper limb spasticity, predefined movements may include wrist pronation to isolate the pronator teres muscle, while for lower limb spasticity, ankle plantarflexion may be used to target the gastrocnemius muscle. For cervical dystonia, head rotation may be employed to isolate activity in the sternocleidomastoideole. In subjects presenting with muscle spasticity or hypertonicity, a clinical protocol may be implemented to induce a spastic muscle response through positional manipulation, passive stretching, or application of resistance, thereby enhancing the detectability of pathological motor unit activity. During these activation protocols, the electrodes 106 detect electrical potentials generated by motor unit action potentials propagating along muscle fibers, and these signals are recorded continuously across all channels of the electrode array 102. The high-density spatial sampling provided by the electrode array 102 enables the system to capture fine spatial variations in motor unit activity, thereby providing the resolution necessary to distinguish between discrete motor unit territories within the target muscle.

At step 206, the electromyographic signals are transmitted from the electrode array 102 to a signal acquisition and therapy module 110. The electrode array 102 may be connected to the signal acquisition and therapy module 110 via a wired connection 108, such as a shielded multiconductor cable, or alternatively via a wireless connection using protocols such as Bluetooth® or Wi-Fi® as described with reference to FIGS. 13-15. The signal acquisition and therapy module 110 receives the raw electromyographic signals and performs initial conditioning operations, including analog amplification via an analog front end configured to provide high-gain, low-noise amplification of bioelectric signals in the microvolt range. Following amplification, the signals are digitized using an analog-to-digital converter (ADC) operating at a sampling rate sufficient to capture the frequency content of motor unit action potentials, such as 2048 Hz or higher. The digitized signals are then transmitted to a processor 114, which may be implemented in a computing device such as a laptop computer, tablet computer, or dedicated processing unit, via a network 112 that may be a wired or wireless communication link. The transmission of digitized EMG data from the signal acquisition and therapy module 110 to the processor 114 enables real-time or near-real-time analysis of neuromuscular activity, supporting the subsequent steps of the method 200.

At step 208, the electromyographic signals are processed to identify motor unit territories within the target muscle. The processor 114 applies digital signal processing techniques to the acquired EMG data, including band-pass filtering to isolate relevant electromyographic frequencies (e.g., 10 Hz to 500 Hz using a second-order Butterworth filter) and notch filtering to attenuate mains interference (e.g., a 60 Hz notch filter). Following preprocessing, the processor 114 applies advanced signal processing algorithms, such as blind source separation techniques, to decompose the composite EMG signals into individual motor unit action potentials (MUAPs). In one embodiment, the system employs independent component analysis (ICA) or FastICA algorithms to unmix overlapping signals and isolate individual motor unit contributions. The processor 114 computes projections of weight matrices derived from the blind source separation onto the recorded data, and identifies clusters of recording sites based on the projections, thereby grouping electrodes that detect activity from the same motor unit. The temporal characteristics of the identified clusters are analyzed to determine activation frequency, regularity, and firing patterns, and the identified clusters are mapped to anatomical locations corresponding to motor unit territories within the monitored musculature. Spatial filter banks are constructed based on the identified spatially correlated regions, with each spatial filter bank configured to isolate electromyographic activity associated with a corresponding motor unit territory. These filter banks function as beamforming algorithms that enhance signals originating from specific spatial locations while attenuating signals from other regions, thereby enabling precise localization of motor unit territories and innervation zones within the target muscle.

At step 210, the processor 114 generates spatial maps of neuromuscular activity based on the processed electromyographic signals. In one embodiment, a root mean square (RMS) heatmap is generated by calculating RMS values of the EMG signals recorded from each electrode 106 during resting trials and normalizing these values against the peak RMS values observed during maximum voluntary contraction (MVC) trials, as described with reference to FIG. 10. The resulting resting RMS ratio for each electrode provides a quantitative metric of neuromuscular activity at that spatial location, with elevated ratios indicating regions of high resting activity characteristic of hypertonicity, spasticity, or involuntary muscle activation. The calculated RMS ratios are mapped onto a color-coded or intensity-scaled visualization, such that regions of heightened activity appear as “hot spots” on the heatmap, providing clinicians with spatially resolved guidance for selecting optimal injection sites. In addition to the RMS heatmap, the processor 114 may generate innervation zone (IZ) point clouds, as described with reference to FIG. 11, which provide three-dimensional anatomical coordinates specifying not only the lateral position of motor unit territories on the muscle surface but also the estimated depth of innervation zones below the skin surface. The depth estimation is performed using bioelectric source imaging techniques, in which the amplitude distribution and spatial extent of recorded signals across adjacent electrodes are analyzed to estimate the distance of bioelectric sources from the electrode array 102. The combination of surface-level activity mapping (RMS heatmap) and depth-resolved anatomical coordinates (IZ point clouds) provides comprehensive guidance for therapeutic agent delivery, enabling clinicians to select both the appropriate void 107 for needle insertion and the optimal insertion depth and trajectory for reaching the target motor unit territory.

At step 212, injection coordinates are identified that correspond to voids 107 in the flexible substrate 104 of the electrode array 102. The processor 114 analyzes the spatial maps generated in step 210 to identify specific motor unit territories exhibiting pathological activity patterns, such as elevated resting activity, excessive firing rates, or involuntary activation. For each identified motor unit territory, the processor 114 determines the spatial coordinates on the electrode array 102 that correspond to the location of maximal activity or to the calculated innervation zone location. The processor 114 then identifies one or more voids 107 within the flexible substrate 104 that are spatially aligned with or proximate to the identified motor unit territories. The voids 107 serve as access points through which a syringe needle can be inserted to deliver the therapeutic agent directly to the underlying motor unit territory without the need to reposition or remove the electrode array 102. In embodiments where depth information is available from the IZ point clouds, the processor 114 may further calculate and display the recommended needle insertion angle and depth, thereby providing comprehensive injection guidance that accounts for both the surface location and the three-dimensional anatomical position of the target motor unit territory. The injection coordinates and recommended insertion parameters may be displayed on a user interface of the processor 114 or the computing device 116, allowing the clinician to visualize the injection plan and execute the procedure with precision.

Finally, at step 214, a therapeutic agent is delivered through the voids 107 in the flexible substrate 104 to the identified motor unit territories. The clinician selects a syringe loaded with a neurolytic agent, such as botulinum toxin, and inserts the needle through the void 107 corresponding to the identified injection coordinates. The void 107 provides unobstructed access for the needle to penetrate the substrate 104 and underlying skin, thereby allowing the therapeutic agent to be injected directly at or near the motor unit territory identified through the electromyographic analysis. The voids 107 are configured to inhibit contamination of the injection site by preventing substrate material from being displaced or introduced into the subject's body during the syringe-mediated transfer of the therapeutic agent. By delivering the neurolytic agent directly to motor endplate territories identified through functional EMG analysis, the method 200 maximizes therapeutic precision and efficacy while minimizing the dosage required to achieve clinical outcomes and reducing the risk of off-target effects on adjacent healthy muscle tissue. Following injection, the electrode array 102 may remain in place to allow post-injection verification of treatment efficacy, such as by acquiring additional EMG signals to assess changes in motor unit activity, or the electrode array 102 may be removed and the procedure concluded. In certain embodiments, the method 200 may be repeated at multiple injection sites within the same muscle or across multiple muscle groups during a single therapeutic session, thereby enabling comprehensive treatment of neuromuscular disorders affecting multiple motor unit territories.

FIG. 13 illustrates example components of the signal acquisition and therapy module 110 of FIG. 1, which functions as the processor or computing device of the system 100. As shown, the signal acquisition and therapy module 110 comprises an integrated assembly of analog and digital circuitry configured to condition, amplify, and digitize electromyographic (EMG) signals obtained from the electrode array 102, and to generate and deliver therapeutic stimulation waveforms back through the electrodes 106. The signal acquisition and therapy module 110 serves as an interface between the electrode array 102 and the external processor 114, enabling bidirectional communication for both signal acquisition and therapy delivery. In various embodiments, the signal acquisition and therapy module 110 may be implemented as a compact, portable device suitable for clinical use, home-based therapy, or wearable applications, with form factors ranging from handheld units to body-worn modules integrated with or proximate to the electrode array 102.

The signal acquisition and therapy module 110 includes an analog front end 150, which is configured to receive raw bioelectric signals from the plurality of electrodes 106 and perform initial signal conditioning operations. The analog front end 150 comprises one or more instrumentation amplifiers or operational amplifiers configured to provide high-gain, low-noise amplification of the microvolt-level EMG signals detected by the electrodes 106. In various embodiments, the analog front end 150 may provide differential amplification with common-mode rejection to suppress noise and artifacts originating from external electromagnetic interference, motion artifacts, or electrode-skin interface fluctuations. The analog front end 150 may further include analog filtering stages, such as high-pass filters to remove DC offsets and low-frequency drift, low-pass filters to attenuate high-frequency noise, and notch filters to suppress power line interference at 50 Hz or 60 Hz. In certain embodiments, the analog front end 150 includes programmable gain stages that can be adjusted based on the amplitude of the incoming EMG signals, thereby optimizing the dynamic range and signal-to-noise ratio of the recorded data. The analog front end 150 may further include multiplexing circuitry to sequentially sample signals from the multiple electrodes 106 within the electrode array 102, or may include parallel amplification channels to enable simultaneous acquisition of signals from all electrodes for high-density spatial mapping.

The conditioned analog signals from the analog front end 150 are transmitted to an analog-to-digital converter (ADC) 152, also referred to as a digitizer, which converts the continuous-time analog signals into discrete-time digital data streams suitable for digital signal processing. The ADC 152 operates at a sampling rate selected to satisfy the Nyquist criterion for the frequency content of EMG signals, such as 1024 Hz, 2048 Hz, or higher, thereby ensuring that all relevant frequency components of motor unit action potentials are captured without aliasing. In various embodiments, the ADC 152 may have a resolution of 12 bits, 16 bits, 24 bits, or higher, providing sufficient amplitude quantization to preserve the dynamic range and fidelity of the EMG signals. The ADC 152 may be implemented as a multi-channel converter capable of simultaneously digitizing signals from multiple electrodes 106, or as a single-channel converter operating in conjunction with the multiplexing circuitry of the analog front end 150 to sequentially digitize signals from each electrode. The digital output of the ADC 152 is transmitted to the microcontroller unit (MCU) or field-programmable gate array (FPGA) 154 for further processing.

The microcontroller unit (MCU) or field-programmable gate array (FPGA) 154 serves as the digital processing core of the signal acquisition and therapy module 110, executing firmware or hardware logic to manage data acquisition, perform real-time preprocessing, coordinate communication with the external processor 114, and control therapy delivery operations. In embodiments employing an MCU, the microcontroller may include one or more processor cores, on-chip memory (RAM and flash), and peripheral interfaces (UART, SPI, I2C, USB) to support data buffering, protocol handling, and system management functions. In embodiments employing an FPGA, the field-programmable gate array provides reconfigurable hardware logic that can be customized to implement parallel signal processing algorithms, real-time filtering, and low-latency control of therapy waveforms. The MCU or FPGA 154 may perform initial preprocessing of the digitized EMG signals, such as downsampling, decimation, or baseline correction, before transmitting the data to the processor 114 via the communication interface 158. The MCU or FPGA 154 further receives therapy control commands from the processor 114, such as stimulation parameters specifying frequency, amplitude, pulse width, and duty cycle, and coordinates the generation and delivery of therapeutic waveforms through the digital-to-analog converter (DAC) 160 and current/voltage driver circuit 162.

The signal acquisition and therapy module 110 further includes a power supply 156, which provides regulated electrical power to all components within the module. The power supply 156 may comprise a rechargeable battery pack, such as a lithium-ion or lithium-polymer battery, dimensioned to provide sufficient energy capacity for extended therapy sessions or multiple uses between recharging cycles. In certain embodiments, the power supply 156 includes voltage regulation circuitry to provide stable supply voltages for the analog front end 150, ADC 152, MCU or FPGA 154, and driver circuits, thereby ensuring consistent performance and signal fidelity across varying battery charge levels. The power supply 156 may further include power management circuitry to monitor battery status, implement low-power sleep modes during periods of inactivity, and provide charging circuitry for recharging the battery via a USB port, wireless inductive coupling, or other charging interface. In alternative embodiments, the power supply 156 may comprise disposable batteries, such as AA or AAA cells, or may be powered via an external AC adapter or tethered connection to the processor 114.

The communication interface 158 enables bidirectional data transfer between the signal acquisition and therapy module 110 and the external processor 114 via the network 112. In embodiments employing wireless communication, the communication interface 158 may comprise a Bluetooth® transceiver, Wi-Fi® radio, Zigbee® module, or other short-range or long-range wireless protocol, thereby allowing the electrode array 102 and signal acquisition and therapy module 110 to be positioned on the patient without physical tethering to the processor 114. The wireless communication interface 158 may implement standard protocols, such as Bluetooth® Low Energy (BLE) for power-efficient data streaming, or may employ proprietary protocols optimized for low-latency, high-bandwidth transmission of multi-channel EMG data and therapy control commands. In embodiments employing wired communication, the communication interface 158 may comprise a USB port, Ethernet connector, or dedicated data cable interface, providing reliable, high-speed data transfer with minimal latency. The communication interface 158 handles packetization, error checking, and retransmission of data to ensure robust communication even in noisy or interference-prone environments.

In embodiments configured to deliver electrical stimulation therapy, the signal acquisition and therapy module 110 further includes a digital-to-analog converter (DAC) 160 and a current/voltage driver circuit 162. The DAC 160 receives digital waveform data from the MCU or FPGA 154 specifying the desired therapeutic stimulation waveform, such as high-frequency blocking stimulation (1 kHz to 10 kHz) for muscle relaxation or modulated high-frequency stimulation with a low-frequency envelope (10 Hz to 50 Hz modulation) for muscle strengthening, and converts the digital waveform into a continuous-time analog signal. The current/voltage driver circuit 162 amplifies and conditions the analog signal from the DAC 160, generating the appropriate current or voltage levels required to deliver the therapeutic waveform through selected electrodes 106 within the electrode array 102. The driver circuit 162 may include current-limiting circuitry, impedance monitoring, and safety shutoff mechanisms to ensure that stimulation parameters remain within safe therapeutic ranges and to prevent tissue damage or patient discomfort. In certain embodiments, the driver circuit 162 includes multiplexing or switching circuitry to selectively route stimulation signals to specific electrodes 106 based on the identified motor unit territories, thereby enabling spatially targeted therapy delivery. By integrating both signal acquisition and therapy delivery capabilities within a single module 110, the system 100 enables closed-loop neuromuscular modulation with minimal hardware complexity and improved spatial alignment between sensing and stimulation electrodes.

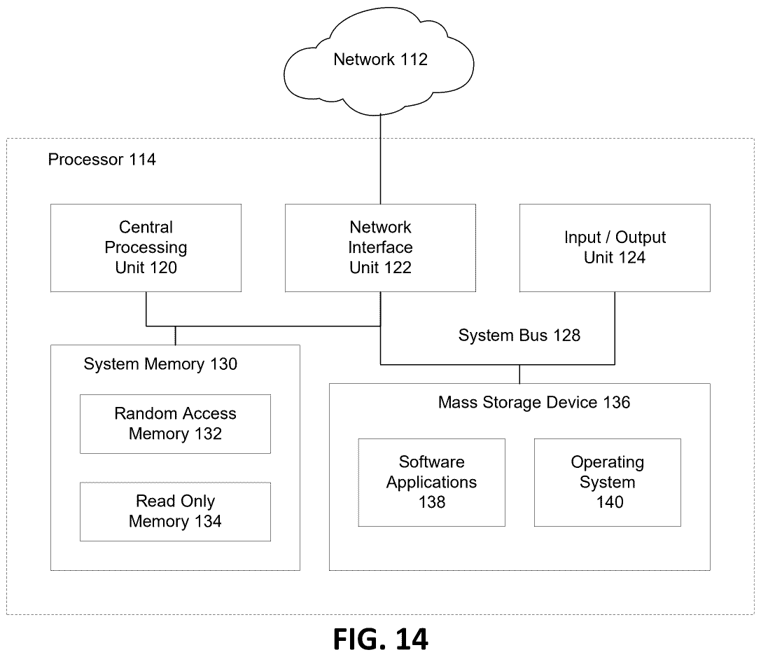

FIG. 14 is an illustration of an embodiment of the processor 114 or computing device of the system 100 configured to provide processing and control functionality. As shown, the processor 114 comprises a general-purpose computing architecture capable of executing software instructions to perform the signal processing, motor unit identification, spatial mapping, and therapeutic decision-making functions described throughout this disclosure. The processor 114 may be implemented as a laptop computer, desktop workstation, tablet computer, smartphone, or dedicated embedded processing unit, and serves as the central computational platform for the neuromuscular modulation system 100. In various embodiments, the processor 114 receives digitized electromyographic (EMG) data from the signal acquisition and therapy module 110 via the network 112, processes the data to identify motor unit territories and innervation zones, generates spatial maps such as RMS heatmaps and innervation zone point clouds, and determines therapeutic stimulation parameters that are communicated back to the signal acquisition and therapy module 110 for delivery through the electrode array 102.

The processor 114 includes at least one central processing unit (CPU) 120, which executes machine-readable instructions to perform computational operations required for signal processing, data analysis, and control functions. The CPU 120 may be a multi-core processor, such as a dual-core, quad-core, or higher core-count processor, enabling parallel execution of multiple processing threads to improve computational throughput and reduce latency in real-time signal processing applications. In various embodiments, the CPU 120 may be supplemented or replaced by specialized processing units, such as graphics processing units (GPUs) for parallel computation of filtering operations, digital signal processors (DSPs) optimized for mathematical operations on signal data, or application-specific integrated circuits (ASICs) configured to accelerate specific algorithms such as blind source separation or motor unit decomposition. The CPU 120 is operably connected to other system components via a system bus 128, which provides a communication pathway for data transfer between the CPU 120, system memory 130, mass storage device 136, and peripheral interfaces.

The system memory 130 comprises volatile and non-volatile memory components that store data and instructions required for operation of the processor 114. As shown in FIG. 14, the system memory 130 includes random access memory (RAM) 132 and read only memory (ROM) 134. The RAM 132 provides high-speed, volatile storage for data and program instructions that are actively being executed by the CPU 120, including buffered EMG signal data, intermediate computation results, spatial filter coefficients, motor unit territory maps, and real-time processing variables. The RAM 132 may be implemented using dynamic RAM (DRAM) technology, such as DDR4 or DDR5 memory modules, with capacities ranging from several gigabytes to tens of gigabytes or more, depending on the computational requirements of the signal processing algorithms and the number of electrode channels being processed simultaneously. The ROM 134 provides non-volatile storage for firmware and basic input/output system (BIOS) routines that initialize the processor 114 during startup and manage low-level hardware interfaces. In certain embodiments, the ROM 134 may be implemented as flash memory or electrically erasable programmable read-only memory (EEPROM), allowing for firmware updates and configuration changes while retaining essential boot-level functionality.

The processor 114 further includes a mass storage device 136, which provides non-volatile, persistent storage for software applications 138, operating system 140, and data files. The mass storage device 136 may be implemented as a hard disk drive (HDD), solid-state drive (SSD), hybrid drive, or other non-transitory computer-readable storage medium. In various embodiments, the mass storage device 136 stores the operating system 140, such as Windows®, macOS®, Linux®, Android®, iOS®, or another suitable operating system platform, which manages hardware resources, provides application programming interfaces (APIs) for software applications, and coordinates multitasking and process scheduling. The mass storage device 136 also stores software applications 138, which include the signal processing algorithms, motor unit decomposition routines, spatial mapping functions, user interface software, and therapy control logic specific to the neuromuscular modulation system 100. The software applications 138 may be developed using programming languages such as C, C++, Python, Java, or MATLAB®, and may incorporate libraries or frameworks for digital signal processing (e.g., FFTW, SciPy), machine learning (e.g., TensorFlow, PyTorch), and graphical user interface development (e.g., Qt, Electron). The mass storage device 136 further stores patient data files, including recorded EMG sessions, motor unit territory maps, therapy session logs, treatment outcomes, and configuration profiles for individual patients or clinical protocols.

Communication between the processor 114 and external devices, such as the signal acquisition and therapy module 110, is facilitated by a network interface unit 122. The network interface unit 122 provides connectivity through wired or wireless communication protocols, enabling bidirectional data transfer between the processor 114 and the signal acquisition and therapy module 110 via the network 112. In embodiments employing wired communication, the network interface unit 122 may comprise a USB controller, Ethernet adapter, or serial port interface, providing reliable, high-bandwidth data transfer with low latency and minimal susceptibility to interference. In embodiments employing wireless communication, the network interface unit 122 may comprise a Bluetooth® transceiver for short-range communication, a Wi-Fi® radio for local area network connectivity, or a cellular modem for wide-area network access, thereby enabling untethered operation of the electrode array 102 and signal acquisition and therapy module 110 while maintaining real-time communication with the processor 114. The network interface unit 122 handles protocol stack operations, including packetization, error detection and correction, flow control, and retransmission, ensuring robust communication even in challenging electromagnetic environments such as clinical settings with medical imaging equipment or other sources of radio frequency interference.

The processor 114 also includes an input/output (I/O) unit 124, which manages user interaction and peripheral device connectivity. The I/O unit 124 may be coupled to input devices such as a keyboard, mouse, touchscreen display, trackpad, stylus, or voice recognition interface, enabling clinicians or patients to interact with the software applications 138 to initiate EMG recording sessions, review spatial maps, adjust therapy parameters, annotate patient records, or configure system settings. The I/O unit 124 may further be coupled to output devices such as a graphical display screen, audio speakers, haptic feedback actuators, or printers, enabling the processor 114 to present visual feedback (e.g., RMS heatmaps, innervation zone point clouds, user interface elements), auditory cues (e.g., alerts, prompts, progress indicators), tactile feedback (e.g., vibration patterns to guide user actions), or printed reports (e.g., therapy session summaries, injection coordinate tables). In embodiments where the processor 114 is implemented as a touchscreen tablet or smartphone, the I/O unit 124 manages the touch-sensitive display interface, enabling intuitive, gesture-based interaction with the neuromuscular modulation system 100 and supporting portable, bedside, or at-home use cases.

The system bus 128 serves as the primary communication backbone within the processor 114, connecting the CPU 120, system memory 130, mass storage device 136, network interface unit 122, and I/O unit 124. The system bus 128 may be implemented using industry-standard bus architectures, such as PCI Express (PCIe), USB, or proprietary high-speed interconnects, and provides data transfer rates sufficient to support real-time processing of multi-channel EMG data streams, typically ranging from hundreds of megabytes per second to several gigabytes per second depending on the number of electrode channels and the sampling rate employed. By providing a unified communication pathway, the system bus 128 enables efficient coordination of data flow between hardware components, minimizes latency in signal processing pipelines, and supports the concurrent execution of multiple processing tasks required for closed-loop neuromuscular modulation.

During operation, the processor 114 executes the software applications 138 stored on the mass storage device 136, loading program instructions and data into the RAM 132 for high-speed access by the CPU 120. The software applications 138 receive digitized EMG data from the signal acquisition and therapy module 110 via the network interface unit 122, apply digital signal processing algorithms such as band-pass filtering, notch filtering, and motor unit decomposition to identify discrete motor unit action potentials, compute spatial maps including RMS heatmaps and innervation zone point clouds to visualize neuromuscular activity, analyze the spatial and temporal characteristics of motor unit activity to classify muscle regions as hypertonic or hypotonic, and generate therapeutic stimulation parameters tailored to the identified neuromuscular state. The processor 114 then transmits the stimulation parameters back to the signal acquisition and therapy module 110 via the network interface unit 122, enabling closed-loop control in which EMG signals guide therapy delivery in real time. By integrating computational processing, data storage, user interface management, and network communication within a single computing device architecture, the processor 114 provides the intelligence and control logic necessary to transform the neuromuscular modulation system 100 from a passive recording device into an adaptive, precision-guided therapeutic platform.

It should be understood that the examples provided above are merely illustrative and are not intended to limit the scope of the present disclosure. Numerous modifications, variations, and equivalents will be apparent to those of skill in the art in view of the teachings herein. For instance, while particular electrode geometries, substrate materials, and stimulation frequencies have been described, other configurations, materials, or parameter ranges may be employed without departing from the spirit of the invention. Similarly, although particular clinical applications such as pelvic floor dysfunction, limb spasticity, and facial dystonia have been discussed, the disclosed system is applicable to a wide range of neuromuscular disorders and therapeutic use cases.

Accordingly, the embodiments disclosed herein should be regarded as exemplary rather than restrictive. The scope of the present invention is defined not by the foregoing description but instead by the appended claims, together with all equivalents that would be understood by one of ordinary skill in the art.

What is claimed is:

1. An electrode array for electromyography-guided therapeutic intervention, comprising:
a flexible substrate fabricated from a biocompatible polymeric material;
a plurality of electrodes disposed on the flexible substrate and arranged in a structured array configuration, wherein the plurality of electrodes are spatially distributed on the flexible substrate in rows and columns at intervals corresponding to a sampling frequency less than or equal to a Nyquist frequency of target muscle signals;
a plurality of conductive traces deposited on or embedded within the flexible substrate, wherein each conductive trace operably connects a corresponding electrode to a connection interface;
a plurality of voids formed within the flexible substrate, the voids extending completely through a thickness of the flexible substrate to provide unobstructed access for syringe-mediated delivery of therapeutic agents to underlying tissue, wherein the plurality of voids are arranged in one or more rows positioned between adjacent rows of electrodes; and
a wired connection interface positioned adjacent to a first edge of the flexible substrate, the wired connection interface configured to operably connect the conductive traces to a signal acquisition and therapy module, wherein the conductive traces extend from the plurality of electrodes to the wired connection interface along the flexible substrate.

2. The electrode array of claim 1, wherein the flexible substrate has a generally rectangular shape with rounded corners.

3. The electrode array of claim 1, wherein the biocompatible polymeric material comprises thermoplastic urethane.

4. The electrode array of claim 1, wherein the structured array configuration comprises at least one of an 8 by 8 array, an 8 by 10 array, or an 8 by 12 array of electrodes.

5. The electrode array of claim 1, wherein each electrode comprises a conductive contact pad constructed of a material including at least one of gold, silver, silver/silver chloride (Ag/AgCl), stainless steel, or platinum/iridium (Pt/Ir).

6. The electrode array of claim 1, wherein each void has a cross-sectional dimension ranging from approximately 1 mm to 5 mm to accommodate passage of a hypodermic needle while minimizing loss of structural integrity of the flexible substrate.

7. The electrode array of claim 1, wherein the flexible substrate further comprises an adhesive backing configured to secure the electrode array to a skin surface.

8. The electrode array of claim 7, wherein the adhesive backing comprises a biocompatible adhesive selected from at least one of a hydrogel adhesive, an acrylic adhesive, or a silicone-based adhesive.

9. The electrode array of claim 1, wherein the conductive traces are fabricated from an electrically conductive material comprising at least one of copper, gold, platinum, or silver.

10. The electrode array of claim 1, wherein the plurality of voids comprises at least 20 voids arranged in the one or more rows between adjacent rows of electrodes.

11. The electrode array of claim 1, wherein the wired connection interface comprises a connector selected from at least one of a snap connector, a zero insertion force (ZIF) connector, or a flexible printed circuit (FPC) connector.

12. The electrode array of claim 1, wherein the flexible substrate has a thickness ranging from approximately 0.1 mm to 2 mm.

13. The electrode array of claim 1, wherein the plurality of electrodes comprises between 64 and 128 electrodes.

14. The electrode array of claim 1, wherein the intervals between adjacent electrodes range from approximately 5 mm to 15 mm.

15. A system for electromyography-guided therapeutic intervention, comprising:
an electrode array comprising:
a flexible substrate fabricated from a biocompatible polymeric material;
a plurality of electrodes disposed on the flexible substrate and arranged in a structured array configuration, wherein the plurality of electrodes are spatially distributed on the flexible substrate in rows and columns at intervals corresponding to a sampling frequency less than or equal to a Nyquist frequency of target muscle signals;
a plurality of conductive traces deposited on or embedded within the flexible substrate, wherein each conductive trace operably connects a corresponding electrode to a connection interface;
a plurality of voids formed within the flexible substrate, the voids extending completely through a thickness of the flexible substrate to provide unobstructed access for syringe-mediated delivery of therapeutic agents to underlying tissue, wherein the plurality of voids are arranged in one or more rows positioned between adjacent rows of electrodes; and
a wired connection interface positioned adjacent to a first edge of the flexible substrate, wherein the conductive traces extend from the plurality of electrodes to the wired connection interface along the flexible substrate; and
a signal acquisition and therapy module operably connected to the electrode array via the wired connection interface, the signal acquisition and therapy module comprising:
an analog front end configured to receive electromyographic signals from the plurality of electrodes and to amplify the electromyographic signals;
an analog-to-digital converter configured to convert the amplified electromyographic signals into digital signals;
a microcontroller unit or field-programmable gate array configured to process the digital signals and to generate therapy control commands; and
a communication interface configured to transmit the digital signals to a processor and to receive therapy parameters from the processor.

16. The system of claim 15, wherein the signal acquisition and therapy module further comprises:
a digital-to-analog converter configured to convert digital therapy waveforms into analog therapy signals; and
a current/voltage driver circuit configured to deliver the analog therapy signals through selected electrodes of the plurality of electrodes.

17. The system of claim 15, wherein the signal acquisition and therapy module further comprises a power supply comprising a rechargeable battery.

18. The system of claim 15, wherein the communication interface comprises a wireless transceiver selected from at least one of a Bluetooth transceiver, a Wi-Fi radio, or a Zigbee module.

19. The system of claim 15, further comprising a processor configured to:
receive the digital signals from the signal acquisition and therapy module;
process the digital signals to identify motor unit territories within a target muscle; and
generate the therapy parameters based on the identified motor unit territories.

20. The system of claim 19, wherein the processor is further configured to generate at least one of a root mean square heatmap or an innervation zone point cloud based on the digital signals.
